(19) United States
(12) Patent Application Publication
(10) Pub. No.: US 2009/0131471 A1
(43) Pub. Date: May 21, 2009

(54) Compositions useful for treating gastroesophageal reflux disease

(75) Inventors: Suhail Nurbhai, Sudbury, MA (US); Steven B. Landau, Wellesley, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Dynogen Pharmaceuticals, Inc.

(21) Appl. No.: 12/156,787

(22) Filed: Jun. 4, 2008

Related U.S. Application Data
(60) Provisional application No. 60/933,535, filed on Jun. 7, 2007.

(51) Int. Cl. A61K 31/439; A61P 1/00

(52) U.S. Cl. 514/305

(57) ABSTRACT

The present invention relates to a method of treating GERD in a human subject in need of treatment. The method comprises orally administering to said subject an effective amount of a thieno[3,2-b]pyridine compound of Structural Formula I or a pharmaceutically acceptable salt or N-oxide derivative thereof, wherein the effective amount is from about one to about three daily doses of the compound and the dose is from about 0.2 mg to about 0.5 mg.

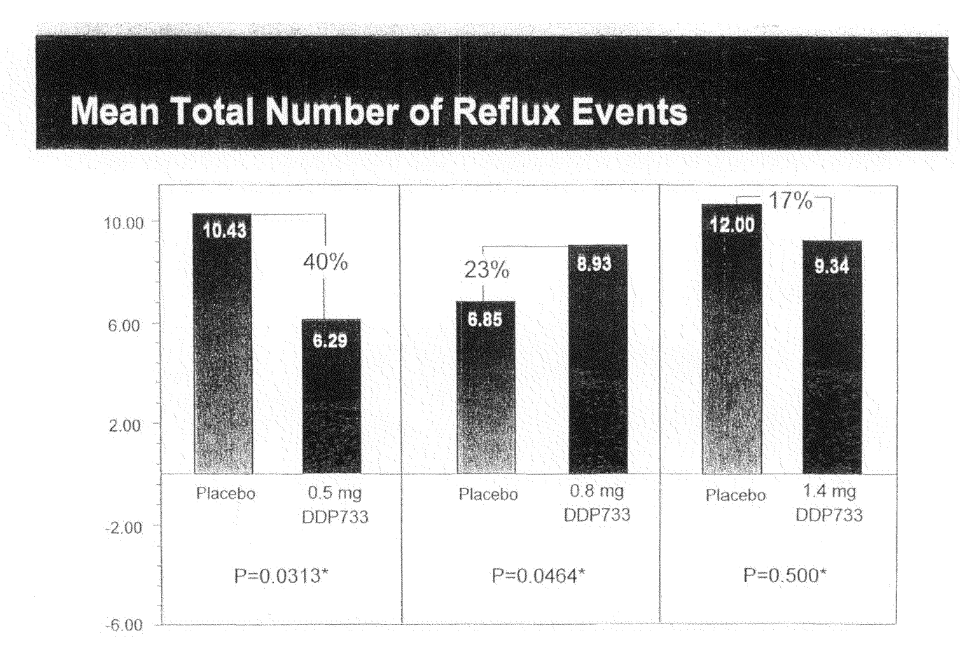

### RELATED APPLICATION

This application claims the benefit of U.S. Provisional Application No. 60/933,535, filed on Jun. 7, 2007. The entire teachings of the above application are incorporated herein by reference.

### BACKGROUND OF THE INVENTION

Gastroesophageal reflux is a physical condition in which stomach contents (e.g, stomach acid) reflux or flow back from the stomach into the esophagus. Frequent reflux episodes (e.g., two or more times per week) can result in a more severe problem known as gastroesophageal reflux disease (GERD). The most common symptom of GERD is a burning sensation or discomfort behind the breastbone or sternum and is referred to as dyspepsia or heartburn. Dyspepsia can also mimic the symptoms of myocardial infarction or severe angina pectoris. Other symptoms of GERD include dysphagia, odynophagia, hemorrhage, water brash and respiratory manifestations such as asthma, recurrent pneumonia, chronic coughing, intermittent wheezing due to acid aspiration and/or stimulation of the vagus nerve, earache, hoarseness, laryngitis and pharyngitis.

Reflux episodes which result in GERD, can occur in sufferers during the daytime (i.e., when the subject is in a waking state), at nighttime (i.e., when the subject is in a non-waking state) or at both times (combination refluxers). GERD occurring at nighttime is referred to as nocturnal GERD. As such, there are three distinct patient populations of GERD sufferers: daytime (or diurnal) GERD sufferers; nighttime or nocturnal GERD sufferers and combination GERD sufferers (i.e., both nighttime and daytime).

Nocturnal GERD is distinct from daytime or diurnal GERD not only in the timing of the reflux episode, but in the severity of the damage which occurs as a result of the reflux. More specifically, nocturnal GERD, can be particularly damaging to the pharynx and larynx and a strong association between nocturnal GERD and asthma exists. The increased damage associated with nocturnal GERD is due to a decrease in natural mechanisms which normally help protect against reflux (e.g., saliva production and swallowing), which occur when the patient is sleeping. This decrease leaves the esophagus more vulnerable to damage and can increase microaspiration. In addition, while asleep the body is in the recumbent position, eliminating the effect of gravity, which can clear gastric content from the esophagus. Sleep disorders are also associated with nocturnal GERD resulting in daytime sleepiness and a significant decrease in the overall quality of life.

On a chronic basis, GERD subjects the esophagus to ulcer formation or esophagitis and can result in more severe complications such as, esophageal erosion, esophageal obstruction, significant blood loss and perforation of the esophagus. Severe esophageal ulcerations occur in 20-30% of patients over age 65. In addition to esophageal erosion and ulceration, prolonged exposure of the esophageal mucosa to stomach acid can lead to a condition known as Barrett's Esophagus. Barrett's Esophagus is an esophageal disorder that is characterized by replacement of normal squamous epithelium with abnormal columnar epithelium. This change in tissue structure is clinically important not only as an indication of severe reflux, but as an indication of cancer.

Many factors are believed to contribute to the onset of GERD. A number of factors involve failure of the lower esophageal sphincter (LES) mechanism to work properly. These factors include, for example, increased transient lower esophageal sphincter relaxations (TLESR) and decreased lower esophageal sphincter (LES) resting tone. The LES is a physiologic, non-anatomic area involving the lower 3 centimeters of the esophagus and, like other smooth muscle sphincters in the body (e.g., anal and urinary), the LES is tonically contracted to prevent reflux. In a healthy person the muscle relaxes only during swallowing to allow food to pass and also on average three to four times an hour in a phenomenon known as TLESR. In GERD sufferers, the frequency of TLESR can be much higher, for example, as high as eight or more times an hour and weakness of the LES allows reflux to occur. Other factors which can contribute to GERD include delayed stomach emptying and ineffective esophageal clearance.

Therefore, the extent and severity of GERD depends not only on the presence of gastroesophageal reflux but on factors including the volume of gastric juice available to reflux, the potency of the refluxed material, the interval that the refluxed material remains in the esophagus and the ability of the esophageal tissue to withstand injury and to repair itself after injury.

Current methods to treat GERD include lifestyle changes such as weight loss, avoidance of certain foods that exacerbate the symptoms of GERD and avoidance of excessive bending. Elevation of the head of the bed helps reduce nocturnal reflux. While these avoidance strategies can be useful, the efficacy of lifestyle modification alone for the treatment of GERD is not supported.

Medications for the treatment of GERD include conventional antacids, for example, TUMS® and ROLAIDS® which provide only short term relief. H₂ receptor antagonists, for example, nizatidine (AXID®), ranitidine (ZANTAC®), famotidine (PEPCID® and PEPCID COMPLETE®), roxatidine (ROTANE® or ZORPEX®) and cimetidine (TAGAMET®), are more effective in controlling GERD symptoms, but do not treat the underlying disease. However, patients receiving H₂ receptor antagonists develop tolerance to the drugs rendering the drugs ineffective in their ability to inhibit acid secretion (Fackler et al., Gastroenterology, 122(3):625-632 (2002)).

More powerful secretory inhibitors, such as the proton pump inhibitors, for example, esomeprazole (NEXIUM®), omeprazole (PRILOSEC® and RAPINEX®), lansoprazole (PREVACID®), rabeprazole (PARIET®, ACIPHEX®) and pantoprazole (PROTONIX®) are more effective than the H₂ receptor antagonists but are very expensive and their efficacy relies on inhibition of active proton pumps as stimulated by meals, thereby having little or no effect on the occurrence of nocturnal GERD.

Prokinetic drugs are another type of drug used in the treatment of GERD. Prokinetic drugs act to stimulate gastrointestinal motility. Stimulation can occur by direct action on smooth muscle or by an action on the myenteric plexus. The motor functions of the gastrointestinal tract are expressions of a balance at the level of smooth muscle cells between inhibitory mechanisms mainly regulated by dopamine and stimulatory events mainly regulated through the release of acetylcholine. Therefore gastrointestinal motility can be stimulated by dopamine antagonists such as metoclopramide and domperidone, or by substances which release acetylcholine such as metoclopramide or the 5-HT₄ receptor agonist, cisapride (PROPULSID®), or directly by cholinergic drugs which bind on muscarinic receptors of the smooth muscle cell such as bethanechol. Prokinetic drugs can both stimulate motility and coordinate the activity between different segments of the gastrointestinal tract.

Currently there are no prokinetic drugs available on the market which are both effective and safe. For example, serious cardiac arrhythmias including ventricular tachycardia, ventricular fibrillation, torsades de pointes, and QT prolongation have been reported in patients taking the prokinetic of choice, cisapride. As a result, strict limitations have been imposed on the prescribing of this drug. Further, the use of the dopamine antagonists, metoclopramide and domperidone, is associated with lack of patient tolerability, undesirable CNS effects, such as dyskinesia and undesirable cardiovascular effects, such as QT prolongation.

Other prokinetic agents described in the literature for use in GERD include pumosetrag (CAS Number: 194093-42-0), which is also known as DDP733 (Dynogen Development Program 733) and MKC-733. For example, U.S. Pat. No. 6,967,207 to Yamazaki reports that pumosetrag has gastric acid suppressing activity in addition to its prokinetic activity, making it advantageous in the treatment of GERD. Other literature (e.g., Coleman, N. S. et al. Effect of a Novel 5-HT3 Receptor Agonist MKC-733 on Upper Gastrointestinal Motility in Humans. Aliment Pharmacol Ther 2003; 18(10): 1039-1048), however, reports that pumosetrag delays gastric emptying and relaxes the fundus at a 4.0 mg dose (both undesirable effects in the treatment of GERD), but shows no significant effect on increasing gastric emptying or the fundus at a 0.2 mg and 1.0 mg dose (an increase in gastric emptying is a desirable effect in treating GERD). As such, taking the literature as a whole one would expect that a suitable dose range for the treatment of GERD would be greater than 1.0 mg (in order to achieve an increase in gastric emptying) and less than 4.0 mg per dose (to avoid fundic relaxation and delayed gastric emptying).

In view of the above, better treatment options for GERD, in particular n-GERD, are needed.

### SUMMARY OF THE INVENTION

The present invention is based on the discovery that doses of DDP733, which are significantly less than 1 mg per dose, are particularly useful in the treatment of GERD.

The present invention relates to a method of treating GERD in a subject in need of treatment. The method comprises administering to said subject an effective amount of a thieno[3,2-b]pyridine compound of Structural Formula I or a pharmaceutically acceptable salt (e.g., DDP733) or N-oxide derivative thereof. In a particular embodiment the GERD is n-GERD.

More specifically, the invention relates to a method of treating GERD in a subject in need thereof comprising administering to said subject an effective amount of a compound represented by Structural Formula I:

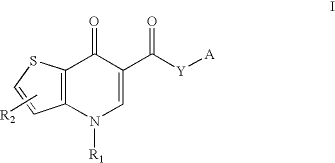

wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or

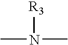

wherein R₃ represents hydrogen or a C₁-C₆ alkyl group; and
A is represented by

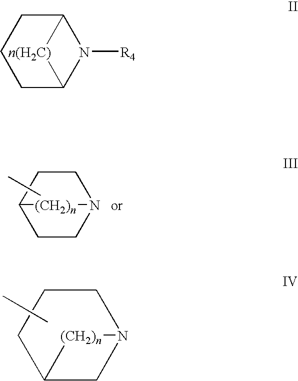

wherein:
n is an integer from 1 to about 4; R₄ represents hydrogen, a C₁-C₆ alkyl group, a C₃-C₈ cycloalkyl group or a C₇-C₁₈ aralkyl group or a pharmaceutically acceptable salt or N-oxide derivative thereof,

wherein the effective amount is from about one to about three daily doses of the compound and the dose is from about 0.2 mg to about 0.5 mg.

In a specific embodiment of Structural Formula I, Y represents —O— or

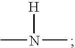

R₁, represents hydrogen, a C₁-C₆ alkyl group, a C₆-C₁₂ aryl group, or a C₇-C₁₈ aralkyl group; R₂ represents hydrogen, a C₁-C₆ alkyl group or halogen; and
A is represented by

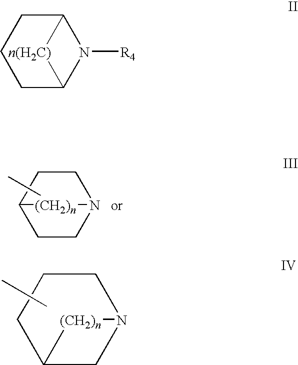

wherein:
   n is 2 or 3; and R₄ represents a C₁-C₆ alkyl group.

In a more specific embodiment, the compound for use in the invention is represented by Structural Formula I, wherein R₁ represents hydrogen or a C₁-C₃ alkyl group; R₂ represents hydrogen, a C₁-C₃ alkyl group or halogen; R₃ represents hydrogen; R₄ represents a C₁-C₃ alkyl group and n is an integer of 2 or 3.

In another specific embodiment, the 5-HT₃ receptor agonist is represented by Structural Formula V:

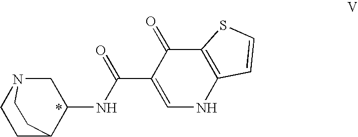

or a pharmaceutically acceptable salt thereof.

In a more specific embodiment, the compound of Structural Formula V has the (R) configuration at the chiral carbon atom which is designated with an asterisk (*). The chemical name of the compound set forth in Structural Formula V having the (R) configuration at the designated chiral carbon is: (R)—N-1-azabicyclo[2.2.2]oct-3-yl-4,7-dihydro-7-oxothieno[3,2-b]pyridine-6-carboxamide.

In a most specific embodiment, the (R)—N-1-azabicyclo[2.2.2]oct-3-yl-4,7-dihydro-7-oxothieno[3,2-b]pyridine-6-carboxamide is in the form of the monohydrochloride, and can be referred to as MKC-733, Dynogen Development Program 733 (DDP733) and pumosetrag (CAS Number: 194093-42-0).

In another embodiment, administration is oral.

In another embodiment, the subject is a human. In a specific embodiment, the human subject is a female. In another specific embodiment, the human subject is a male.

In a further embodiment, the compound is administered in a single daily dose of from about 0.2 to about 0.5 mg. In a yet further embodiment, the dose is about 0.5 mg.

The invention also provides a method of treating GERD in a human subject in need of treatment comprising orally administering to the subject an effective amount of a compound represented by the following structure:

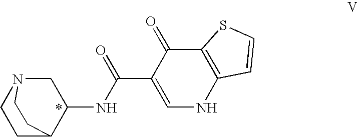

or a pharmaceutically acceptable salt thereof, wherein the effective amount is from about one to about three daily doses of the compound and the dose is from about 0.2 mg to about 0.5 mg.

In a specific embodiment, the asterisked carbon atom of the administered compound is in the (R) configuration. In a more specific embodiment, the compound having the (R) configuration is in the form of the monohydrochloride salt.

The invention also provides a method of treating GERD in a human subject in need of treatment comprising orally administering to the subject an effective amount of DDP733, wherein the effective amount is from about one to about three daily doses of the compound and the dose is from about 0.2 mg to about 0.5 mg. In a particular embodiment, the effective amount is one daily dose of the compound and the dose is from about 0.2 mg to about 0.5 mg. In a more particular embodiment, the effective amount is one daily dose of DDP733 and the dose is about 0.5 mg. In an even more particular embodiment, the effective amount is one daily dose of DDP733, the dose is about 0.5 mg and the subject is suffering from n-GERD.

In a particular embodiment of the method of the invention, the subject is suffering from n-GERD.

In one embodiment, the compounds described herein (e.g., the compounds of Formula V) are administered in a single daily dose of from about 0.2 mg to about 0.5 mg. In a particular embodiment, the single dose is about 0.5 mg. In a specific embodiment, the single dose is administered coincident with the subject's bedtime. In another embodiment, an acid suppressing agent is co-administered with the single daily dose. In a particular embodiment, the acid suppressing agent is a proton pump inhibitor (PPI). In a specific embodiment, the proton pump inhibitor can be selected from the group consisting of: esomeprazole, omeprazole, lansoprazole, rabeprazole and pantoprazole. In a more particular embodiment, the proton pump inhibitor is co-administered with the single dose of from about 0.2 mg to about 0.5 mg (e.g, about 0.5 mg) that is administered coincident with the subject's bedtime (i.e., in the period between the subject's last meal of the day and the subject's bedtime).

In another embodiment, the compounds described herein are administered twice or three times a day. For example, administration is two or three times per day of from about 0.2 mg to about 0.5 mg per each administration. In a specific embodiment, the amount of about 0.5 mg is administered three times a day for a daily total of about 1.5 mg. In a more specific embodiment, the three times a day dosing is coincident with the subject's morning meal, coincident with the subject's midday meal and coincident with the subject's bedtime. In yet another embodiment, an acid suppressing agent is co-administered with one or all doses of the two or three daily doses. In a specific embodiment the acid suppressing agent is a proton pump inhibitor (PPI). In a specific embodiment, the proton pump inhibitor can be selected from the group consisting of: esomeprazole, omeprazole, lansoprazole, rabeprazole and pantoprazole.

Coincident with the morning meal or midday meal of the subject includes up to two hours before commencing the meal or two hours after finishing the meal. Coincident with the subject's bedtime includes the period between the subject's last meal of the day and the subject's bedtime.

The invention further relates to the use of a compound described herein (e.g. a compound of Structural Formula I such as DDP733) for the manufacture of a medicament for treating GERD, in particular n-GERD, in a human subject in need of treatment wherein the medicament is in unit dosage form for oral administration and comprises from about 0.2 mg about 0.5 mg of the compound. In a particular embodiment, the compound of Structural Formula I is DDP733. In an even more particular embodiment, the DDP773 is present at about 0.5 mg.

The invention further relates to a pharmaceutical composition useful for treating GERD in a subject in need of treatment. The pharmaceutically composition comprises from about 0.2 mg to about 0.5 mg of a compound described herein (e.g., a compound of Structural Formula I, such as DDP733) and a pharmaceutically acceptable carrier. In a particular embodiment, the compound of the pharmaceutical composition is DDP733. In an even more particular embodiment, the amount of DDP733 is about 0.5 mg.

### BRIEF DESCRIPTION OF THE DRAWINGS

The FIGURE is a graph showing the mean total number of reflux events measured using multichannel intraluminal impedance for subjects receiving 0.5 mg, 0.8 mg and 1.4 mg of DDP733 and placebo following ingestion of a refluxogenic meal.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

### DETAILED DESCRIPTION OF THE INVENTION

A description of example embodiments of the invention follows.

The thieno[3,2-b]pyridine compounds of Structural Formula I are described in U.S. Pat. No. 5,352,685, the entire content of which is incorporated herein by reference. The thieno[3,2-b]pyridine derivative compounds of Structural Formula I are known to possess 5-HT₃ receptor agonist activity.

### 5-HT₃ Receptor Agonists

The neurotransmitter serotonin was first discovered in 1948 and has been subsequently the subject of substantial scientific research. Serotonin, also referred to as 5-hydroxytryptamine (5-HT), acts both centrally and peripherally on discrete 5-HT receptors. Currently, at least fourteen subtypes of serotonin receptors are recognized and delineated into seven families, 5-HT₁ through 5-HT₇. These subtypes share sequence homology and display some similarities in their specificity for particular ligands. While these receptors all bind serotonin, they initiate different signaling pathways to perform different functions. For example, serotonin is known to activate submucosal intrinsic nerves via 5-HT_{1P} and 5-HT₄ receptors, resulting in, for example, the initiation of peristaltic and secretory reflexes. However, serotonin is also known to activate extrinsic nerves via 5-HT₃ receptors, resulting in, for example, the initiation and perception of unpleasant bowel sensations, including nausea, bloating and pain. A review of the nomenclature and classification of the 5-HT receptors can be found in Neuropharm., 33: 261-273 (1994) and Pharm. Rev., 46:157-203 (1994).

5-HT₃ receptors are ligand-gated ion channels that are extensively distributed on enteric neurons in the human gastrointestinal tract, as well as other peripheral and central locations. Activation of these channels and the resulting neuronal depolarization has been found to affect the regulation of visceral pain and colonic transit. Antagonism of the 5-HT₃ receptors has the potential to influence sensory and motor function in the gut.

As used herein, 5-HT₃ receptor refers to naturally occurring 5-HT₃ receptors (e.g., mammalian 5-HT₃ receptors (e.g., human (Homo sapiens) 5-HT₃ receptors, murine (e.g., rat, mouse) 5-HT₃ receptors, feline (e.g., cat) 5-HT₃ receptors)) and to proteins having an amino acid sequence which is the same as that of a corresponding naturally occurring 5-HT₃ receptor (e.g., recombinant proteins). The term includes naturally occurring variants, such as polymorphic or allelic variants and splice variants.

As used herein, the term a 5-HT₃ receptor agonist refers to a substance (e.g., a molecule, a compound) which promotes (induces or enhances) at least one function characteristic of a 5-HT₃ receptor. In one embodiment, the 5-HT₃ receptor agonist binds the 5-HT₃ receptor (i.e., is a 5-HT₃ receptor agonist). In certain embodiments, the agonist is a partial agonist. Partial agonist, as used herein, refers to an agonist which no matter how high of a concentration is used, is unable to produce maximal activation of the 5-HT₃ receptor. A 5-HT₃ receptor agonist (e.g., a 5-HT₃ receptor agonist) can be identified and activity assessed by any suitable method. For example, the binding affinity of a 5-HT₃ receptor agonist to the 5-HT₃ receptor can be determined by the ability of the compounds to displace [³H]granisetron from rat cortical membranes (Cappelli et al., J. Med. Chem., 42(9): 1556-1575 (1999)). In addition, the agonist activity of the compounds can be assessed in vitro on, for example, the 5-HT₃ receptor-dependent [¹⁴C]guanidinium uptake in NG 108-15 cells as described in Cappelli et al.

The thieno[3,2-b]pyridine derivative compounds suitable for use in the present invention are represented by Structural Formula I:

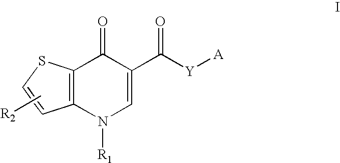

wherein:
R₁ represents hydrogen, a C₁-C₆ alkyl group, a C₂-C₆ alkenyl group, a C₂-C₆ alkynyl group, a C₃-C₈ cycloalkyl group, a C₆-C₁₂ aryl group or a C₇-C₁₈ aralkyl group;
R₂ represents hydrogen, a C₁-C₆ alkyl group, halogen, hydroxyl, a C₁-C₆ alkoxy group, amino, a C₁-C₆ alkylamino group, nitro, mercapto or a C₁-C₆ alkylthio group;
Y represents —O— or

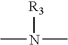

wherein R₃ represents hydrogen or a C₁-C₆ alkyl group; and
A is represented by

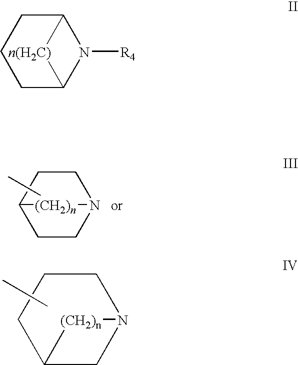

wherein:
n is an integer from 1 to about 4; R₄ represents hydrogen, a C₁-C₆ alkyl group, a C₃-C₈ cycloalkyl group or a C₇-C₁₈ aralkyl group or a pharmaceutically acceptable salt thereof.

It is understood that when R₁ of Structural Formula I is hydrogen, compounds having the tautomeric form represented by Structural Formula IA are included within the definition of Structural Formula I.

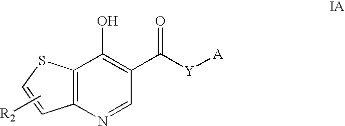

Likewise, it is understood that Structural Formula IA includes the tautomeric form represented by Structural Formula I when R₁ is hydrogen.

In one embodiment, the 5-HT₃ receptor agonist represented by Structural Formula I can be N-oxide derivatives.

In another embodiment of Structural Formula I, Y represents —O— or

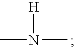

R₁ represents hydrogen, a C₁-C₆ alkyl group, a C₆-C₁₂ aryl group, or a C₇-C₁₈ aralkyl group; R₂ represents hydrogen, a C₁-C₆ alkyl group or halogen; and
A is represented by

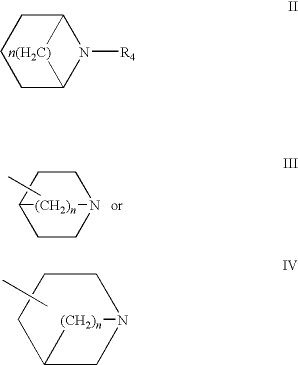

wherein:
   n is 2 or 3; and R₄ represents a C₁-C₆ alkyl group.

In a particular embodiment, the 5-HT₃ receptor agonist is represented by Structural Formula I, wherein R₁ represents hydrogen or a C₁-C₃ alkyl group; R₂ represents hydrogen, a C₁-C₃ alkyl group or halogen; R₃ represents hydrogen; R₄ represents a C₁-C₃ alkyl group and n is an integer of 2 or 3.

In a more particularly embodiment, the 5-HT₃ receptor agonist is represented by Structural Formula V:

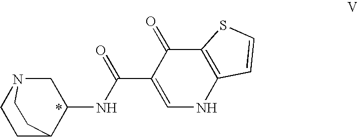

or a pharmaceutically acceptable salt thereof.

In yet another embodiment, the compound represented by Structural Formula V is an N-oxide derivative.

In a most particularly embodiment, the compound of Structural Formula V has the (R) configuration at the chiral carbon atom which is designated with an asterisk (*). The chemical name of the compound set forth in Structural Formula V having the (R) configuration at the designated chiral carbon is: (R)—N-1-azabicyclo[2.2.2]oct-3-yl-4,7-dihydro-7-oxothieno[3,2-b]pyridine-6-carboxamide. When the compound is in the form of the monohydrochloride, it is known as MKC-733, Dynogen Development Program 733 (DDP733) and pumosetrag (CAS Number: 194093-42-0). When the compound of Structural Formula V has the (S) configuration at the chiral carbon atom designated with an asterisk (*), the chemical name is (S)—N-1-azabicyclo[2.2.2]oct-3-yl-4,7-dihydro-7-oxothieno[3,2-b]pyridine-6-carboxamide.

It is understood that Structural Formula V includes the tautomeric form depicted by Structural Formula VA:

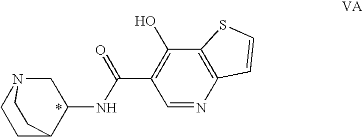

Likewise, it is understood that Structural Formula VA includes the tautomeric form represented by Structural Formula V.

For example, when Structural Formula V has the (R) configuration at the designated chiral carbon the compound is referred to as: (R)—N-1-azabicyclo[2.2.2]oct-3-yl-4,7-dihydro-7-oxothieno[3,2-b]pyridine-6-carboxamide which is understood to include the tautomeric form: (R)—N-1-azabicyclo[2.2.2]oct-3-yl)-7-hydroxythieno[3,2-b]pyridine-6-carboxamide.

Likewise, when Structural Formula VA has the (R) configuration at the designated chiral carbon the compound is referred to as: (R)—N-1-azabicyclo[2.2.2]oct-3-yl)-7-hydroxythieno[3,2-b]pyridine-6-carboxamide, which is understood to include the tautomeric form: (R)—N-1-azabicyclo[2.2.2]oct-3-yl-4,7-dihydro-7-oxothieno[3,2-b]pyridine-6-carboxamide.

As used herein, the term “compound” is intended to include any solvates and hydrates thereof. Thus, it is to be understood that when any compound is referred to by name and structure, solvates and hydrates thereof are included.

### Gastric Acid Suppressing Agents

Gastric acid suppressing agents are agents that suppress gastric acid secretion in the gastrointestinal tract. Agents that act as inhibitors (e.g., antagonists) of any one of the histamine, gastrin or muscarinic receptors present on the surface of parietal cells can suppress gastric acid secretion. Other agents which suppress gastric acid secretion work by inhibiting the enzyme H+-K+ ATPase, commonly referred to as the proton pump, found in parietal cells.

Antagonists of the histamine receptor are commonly referred to as H₂ receptor antagonists and include agents such as cimetidine and ranitidine. Antagonists of the muscarinic receptor include agents such as pirenzepine and propantheline. Antagonists of the gastrin receptor include agents such as proglumide. Inhibitors of H+-K+ ATPase enzyme system include both reversible and irreversible inhibitors such as esomeprazole (NEXIUM®) and soraprazan or AZD0865, respectively.

### Inhibitors of H+-K+ ATPase (Proton Pump)

Inhibitors of H+-K+ ATPase are compounds which can be used to treat gastrointestinal diseases by inhibiting the gastric enzyme H+-K+ ATPase and thereby regulating acidity in gastric juices. More specifically, these inhibitors suppress gastric acid secretion, the final step of acid production, by specific inhibition of H+-K+ ATPase present in gastric parietal cells. Inhibitors of H+-K+ ATPase (proton pump) can bind irreversibly and/or reversibly. Agents referred to as Proton Pump Inhibitors (PPIs) typically include irreversible inhibitors. Agents referred to as Acid Pump Antagonists (APAs) typically include reversible inhibitors.

Proton Pump Inhibitors (PPIs) include benzimidazole compounds, for example, esomeprazole (NEXIUM®), omeprazole (PRILOSEC® and RAPINEX® (oral suspension of omeprazole in combination with an antacid)), lansoprazole (PREVACID®), rabeprazole (PARIET®, ACIPHEX®) and pantoprazole (PROTONIX®). These proton pump inhibitors contain a sulfinyl group situated between substituted benzimidazole and pyridine rings. At neutral pH, esomeprazole, omeprazole, lansoprazole, rabeprazole and pantoprazole are chemically stable, lipid soluble, weak bases that are devoid of inhibitory activity. These uncharged weak bases reach parietal cells from the blood and diffuse into the secretory canaliculi, where the drugs become protonated and thereby trapped. The protonated species rearranges to form a sulfenic acid and a sulfenamide, the latter species capable of interacting with sulfhydryl groups of H+-K+ ATPase. Full inhibition occurs with two molecules of inhibitor per molecule of enzyme. The specificity of the effects of proton pump inhibitors is believed to derive from: a) the selective distribution of H+-K+ ATPase; b) the requirement for acidic conditions to catalyze generation of the reactive inhibitor; and c) the trapping of the protonated drug and the cationic sulfenamide within the acidic canaliculi and adjacent to the target enzyme. Goodman & Gilman's The Pharmacological Basis of Therapeutics, 9^{th} Edition, pp. 901-915 (1996).

The Acid Pump Antagonists (APAs) differ from the PPIs in the way in which they inhibit H+-K+ ATPase. For example, acid induced transformation is not necessary and enzyme kinetics typically show reversible binding to the enzyme for APAs. In addition, APAs can work faster than the PPIs following administration. Suitable APAs include, but are not limited to those described in U.S. Pat. No. 6,132,768 to Sachs et al. and U.S. Published Application No. US2004/0058896 A1 the contents of each of which are incorporated herein by reference. Examples of suitable APAs include, but are not limited to, YH1885 (Yuhan Co.); CS-526 (Sankyo); AZD0865 (AstraZeneca); and Soraprazan (Altana AG).

### H₂ Receptor Antagonists

H₂ receptor antagonists inhibit gastric acid secretion elicited by histamine, other H₂ receptor agonists, gastrin, and, to a lesser extent, muscarinic agonists. H₂ receptor antagonists also inhibit basal and nocturnal acid secretion.

H₂ receptor antagonists competitively inhibit the interaction of histamine with H₂ receptors. They are highly selective and have little or no effect on H₁ receptors. Although H₂ receptors are present in numerous tissues, including vascular and bronchial smooth muscle, they appear to have a minimal role in modulating physiological functions other than gastric acid secretion. H₂ receptor antagonists reduce both the volume of gastric juice secreted and its hydrogen ion concentration. However, despite their good antisecretory properties, H₂ receptor antagonists are not unanimously recognized as gastroprotective agents. H₂ receptor antagonists include nizatidine (AXID®), ranitidine (ZANTAC®), famotidine (PEPCID COMPLETE®, PEPCID®), roxatidine (ROTANE® or ZORPEX®) and cimetidine (TAGAMET®). Goodman & Gilman's The Pharmacological Basis of Therapeutics, 9^{th} Edition, pp. 901-915 (1996). However, patients receiving H₂ receptor antagonists develop tolerance to the drugs rendering the drugs ineffective in their ability to inhibit acid secretion (Fackler et al., Gastroenterology, 122(3):625-632 (2002)).

### Modes of Administration

The compounds for use in the method of the invention can be formulated for oral, transdermal, sublingual, buccal, parenteral, rectal, intranasal, intrabronchial or intrapulmonary administration. Oral administration is preferred. For oral administration the compounds can be of the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY® film coating systems available from Colorcon, West Point, Pa. (e.g., OPADRY®) OY Type, OY-C Type, Organic Enteric OY-P Type, Aqueous Enteric OY-A Type, OY-PM Type and OPADRY® White, 32K18400).

In a particular embodiment, the oral form is a tablet containing DDP733 and the inactive ingredients mannitol, corn starch, microcrystalline cellulose, colloidal silicon dioxide, polyvinyl pyrrolidone, talc, and magnesium stearate, which are coated with an OPADRY® film coating. For example, a 0.5 mg dose of DDP733 can include: 0.5 mg of DDP733; Mannitol 89.9 mg; Corn starch 24.7 mg; Microcrystalline cellulose 6.8 mg; Colloidal silicon dioxide 0.7 mg; Polyvinyl pyrrolidone 2.7 mg; Talc 0.7 mg; Magnesium stearate 4.0 mg; and Opadry white 32K18400 6.5 mg (total core=130.0 mg; total coated 136.5 mg).

Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

For buccal administration, the compounds for use in the method of the invention can be in the form of tablets or lozenges formulated in a conventional manner.

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

For rectal administration, the compounds for use in the method of the invention can be in the form of suppositories or enemas.

For sublingual administration, tablets can be formulated in conventional manner.

For intranasal, intrabronchial or intrapulmonary administration, conventional formulations can be employed.

Further, the compounds for use in the method of the invention can be formulated in a sustained release preparation. For example, the compounds can be formulated with a suitable polymer or hydrophobic material which provides sustained and/or controlled release properties to the active agent compound. As such, the compounds for use the method of the invention can be administered in the form of microparticles for example, by injection or in the form of wafers or discs by implantation.

### Coadministration

An additional therapeutic agent can be used in the method of treating GERD and in compositions of the invention described herein. Additional therapeutic agents suitable for use in the present invention include, but are not limited to, acid suppressing agents (e.g., proton pump inhibitors, H₂ receptor antagonists and acid pump antagonists) and acid neutralizing agents such as antacids, for example, TUMS® and ROLAIDS®. Generally, the additional therapeutic agent will be one that is useful for treating GERD. Preferably, the additional therapeutic agent does not diminish the effects of the therapy and/or potentiates the effects of the primary administration. The therapeutically effective amount of the additional therapeutic agent (e.g., a gastric acid suppressing agent, such as a proton pump inhibitor, an H₂ receptor antagonist or an acid pump antagonist) will depend on the age, sex and weight of the patient, the current medical condition of the patient. The skilled artisan will be able to determine appropriate dosages depending on these and other factors. When the additional therapeutic agent is an approved drug, the generally recommended doses can be used.

When the methods of the invention include coadministration, coadministration refers to administration of a first amount of a compound of Structural Formula I or a pharmaceutically acceptable salt thereof (e.g., DDP733) and a second amount of an additional therapeutic agent. In certain embodiment, the additional therapeutic agent is a gastric acid suppressing agent (e.g., a proton pump inhibitor, an H₂ receptor antagonist or an acid pump antagonist).

Coadministration encompasses administration of the first amount of a compound of Formula I (e.g., DDP733) and an additional therapeutic agent in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When coadministration involves the separate administration of the first amount of the compound of Formula I or a pharmaceutically acceptable salt thereof (e.g., DDP733) and a second amount of an additional therapeutic agent (e.g., a gastric acid suppressing agent such as a proton pump inhibitor, an H₂ receptor antagonist or an acid pump antagonist) the compounds are administered sufficiently close in time to have the desired therapeutic effect. For example, the period of time between each administration, which can result in the desired therapeutic effect, can range from minutes to hours and can be determined taking into account the properties of each compound such as potency, solubility, bioavailability, plasma half-life and kinetic profile.

In a particular embodiment when the coadministration comprises oral administration of a first amount of a compound of Formula I (e.g., DDP733) and a second amount of a gastric acid suppressing agent in a single composition, the gastric acid suppressing agent releases first followed by the compound of Formula I. Release of the agents can occur in the stomach, duodenum or both. For example, a single oral composition can be formulated such that the compound of Formula I (e.g., DDP733) and the gastric acid suppressing agent release in the stomach, duodenum or both. In addition, the composition can be formulated to release the gastric acid suppressing agent first, followed by the compound of Formula I (e.g., DDP733). Staggered release of agents can be accomplished in single composition using any suitable formulation technique such as those described above. For example, a variety of coating thicknesses and/or different coating agents can provide staggered release of agents from a single composition, and release at a desired location in the upper GI tract. In a particular embodiment, a single composition having two portions can be prepared. Portion I can be the gastric acid suppressing agent and portion 2 can be the compound of Formula I (e.g., DDP733). As a first step following administration, the single composition separates into the individual portions. Portion I can begin to release immediately and portion 2 can be formulated to release immediately, release later or release both immediately and later (staggered).

When the coadministration comprises administration of Formula I and a gastric acid suppressing agent as separate compositions, either at the same time or sequentially, the separate compositions can be formulated to achieve the desired release profile. For example, the separate compositions can be formulated to release primarily in the duodenum rather than in the acidic environment of the stomach. In addition, the separate compositions can be formulated such that the gastric acid suppressing agent releases first followed by the compound of Formula I, taking into consideration the amount of time between administration of the separate compositions. A variety of formulation techniques such as gastric retention techniques, coating techniques and the use of suitable excipients and/or carriers can be utilized to achieve the desired release.

### Dosing

The effective amount of the compound of Formula I (e.g., DDP733) can be in the range of from about one to about three daily doses (e.g., two or three daily doses) of the compound, wherein the dose is from about 0.2 mg to about 0.5 mg (e.g., about 0.2 mg, about 0.3 mg, about 0.4 mg or about 0.5 mg). The doses of the compound of Formula I (e.g., DDP733) can be administered at equally spaced intervals in a 24 hour day (e.g., 3 times a day at every 8 hours) or at varying intervals of time during a 24 hour day.

When a single dose is used, the single dose can be administered coincident with the subject's morning meal, coincident with the subject's midday meal or coincident with the subject's bedtime. In a particular embodiment, the single dose is administered coincident with the subject's bedtime. In a more particular embodiment, the single dose is co-administered with an acid suppressing agent (e.g., a proton pump inhibitor). In yet another embodiment, the acid suppressing agent (e.g., a proton pump inhibitor) is co-administered with the single dose of from about 0.2 mg to about 0.5 mg (e.g., 0.5 mg). In a particular embodiment, the acid suppressing agent (e.g., a proton pump inhibitor) is co-administered with the single dose of from about 0.2 mg to about 0.5 mg (e.g., 0.5 mg) that is administered coincident with the subject's bedtime (i.e., in the period between the subject's last meal of the day and the subject's bedtime). In a specific embodiment, the proton pump inhibitor can be selected from the group consisting of: esomeprazole, omeprazole, lansoprazole, rabeprazole and pantoprazole.

When multiple doses are used, the administration is two or three times per day of from about 0.2 mg to about 0.5 mg per each administration. In a specific embodiment, the amount of about 0.5 mg is administered three times a day for a daily total of 1.5 mg. In a more specific embodiment, the three times a day dosing is coincident with the subject's morning meal, coincident with the subject's midday meal and coincident with the subject's bedtime. In yet another embodiment, a proton pump inhibitor (PPI) is co-administered with one or more of the three doses. In a specific embodiment, the proton pump inhibitor can be selected from the group consisting of: esomeprazole, omeprazole, lansoprazole, rabeprazole and pantoprazole.

Coincident with the morning meal or midday meal of the subject includes up to two hours before commencing the meal or two hours after finishing the meal. Coincident with the subject's bedtime includes the period between the subject's last meal of the day and the subject's bedtime.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 2 or 3 times per day). When multiple daily doses are used, the unit dosage form can be the same or different for each dose.

For the compounds of Formula I, each dosage can typically contain from about 0.2 mg to about 0.5 mg. In a preferred embodiment, the compound of Formula I is DDP733 and is present in the unit dosage form about 0.2 mg to about 0.5 mg (e.g., 0.5 mg) in a single dose or in 2 or 3 doses.

It is understood that GERD is synonymous with GORD (gastro-oesophageal reflux disease).

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species. In a particular embodiment, the subject is a human.

As used herein, treating and treatment refer to a reduction in at least one symptom associated with GERD. For example, the subject can experience a reduction in any one or more of the symptoms of heartburn, acid taste, regurgitation, dysphagia, odynophagia, hemorrhage, water brash, esophageal erosion, esophageal obstruction and respiratory manifestations such as asthma, recurrent pneumonia, coughing, intermittent wheezing, earache, hoarseness, laryngitis and pharyngitis.

As used herein, an effective amount refers to an amount sufficient to elicit the desired biological response. In the present invention, the desired biological response is a reduction (complete or partial) of at least one symptom associated with the GERD. As with any treatment, particularly treatment of a multi-symptom disorder, for example, GERD, it is advantageous to treat as many disorder related symptoms which the subject experiences.

The invention further includes a kit for treating GERD, in particular n-GERD. The kit comprises from about one to about three doses of the compound of Formula I wherein each dose is from about 0.2 mg to about 0.5 mg and an instruction insert for administering the compound according to the method of the invention. In a particular embodiment, the compound of Formula I is DDP733. In a most particular embodiment, the kit provides a dose of about 0.5 mg of DDP733 and is for a single daily dose.

As used herein, the term pharmaceutically acceptable salt refers to a salt of the administered compounds prepared from pharmaceutically acceptable non-toxic acids including inorganic acids and organic acids thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

### Clinical Trial

The clinical trial reported here was a Phase 1b, randomized, double-blind, placebo-controlled, crossover study. In the Study, 28 healthy volunteers were administered doses of DDP733 (0.5, 0.8 and 1.4 mg) and placebo and then given a refluxogenic meal. For a period of two hours after completion of the refluxogenic meal, reflux events were measured using intraesophageal impedance.

The results of the clinical trial show that a dose of 0.5 mg of DDP733 achieved statistical significance on the primary clinical endpoint of reduction in the number of reflux events versus placebo. The FIGURE shows that there was a 40% reduction in reflux events for subjects receiving 0.5 mg of DDP733 versus placebo. The 0.8 and 1.4 mg doses did not show a statistically significant reduction in the number of reflux events versus placebo (the FIGURE).

The significant effect achieved at the 0.5 mg dose was unexpected based on the work of Coleman et al. (Coleman, N. S. et al. Effect of a Novel 5-HT3 Receptor Agonist MKC-733 on Upper Gastrointestinal Motility in Humans. Aliment Pharmacol Ther 2003; 18(10): 1039-1048). More specifically, Coleman et al. report that DDP733 delays gastric emptying and relaxes the fundus at a 4.0 mg dose (both undesirable effects in the treatment of GERD), but shows no significant effect on the fundus or increasing gastric emptying at a 0.2 mg and 1.0 mg dose (an increase in gastric emptying is a desirable effect in treating GERD). As such, taking the literature as a whole one would expect that a suitable dose range for the treatment of GERD would be greater than 1.0 mg (in order to achieve an increase in gastric emptying) and less than 4.0 mg per dose (to avoid fundic relaxation and delayed gastric emptying). The inventors of this invention have discovered, however, that a dose significantly less than a dose reported in literature as having no significant effect on gastric emptying or relaxation of the fundus, is particularly useful in the treatment of GERD based on its ability to reduce the number of reflux events.

### Study Details:

Phase 1b, randomized, double-blinded, placebo controlled, crossover study.

28 healthy volunteers

Three Study Periods: Total Number of Visits=4
(1) Ten-day screening period (2 visits);
(2) Two week treatment period (Days 1-14) (2 visits: Day 1 which is on the same day as the second screening visit, and Day 8); and
(3) One day end of study period (1 visit).

Subjects were randomized into a treatment group on Day 1 (the same day as visit 2 of the screening period). For the completed study, each subject was exposed to one of the dose levels of DDP733 (i.e., 0.5 mg, 0.8 mg and 1.4 mg) and to placebo in randomized sequence to complete approximately one week of dosing with each of the two dosing regimens (DDP733 and placebo). The six treatment sequences were:

| Treatment | Dosing Period 1 | Dosing Period 2 |
|---|---|---|
| Sequence | (Days 1-6*) | (Days 8-13*) |
| 1 | Placebo | 0.5 mg DDP733 |
| 2 | 0.5 mg DDP733 | Placebo |
| 3 | Placebo | 0.8 mg DDP733 |
| 4 | 0.8 mg DDP733 | Placebo |
| 5 | Placebo | 1.4 mg DDP733 |
| 6 | 1.4 mg DDP733 | Placebo |

*No dosing on Days 7 and 14

For example, there were 7 subjects in the study who received the 0.5 mg dose of DDP733. At the commencement of dosing period 1, three subjects in the 0.5 mg group received placebo and 4 subjects received 0.5 mg of drug. At the beginning of dosing period 2, the three subjects who already received drug were then administered placebo and the four subjects who received placebo initially, were administered drug. The same randomization was applied to the 0.8 mg group (14 subjects) and the 1.4 mg group (7 subjects).

After randomization, subjects received a first dose of study medication (Day 1). The medication was administered under supervision and the subjects were then evaluated by manometry to assess LES position and pressure and by a multichannel intraluminal impedance and pH (MII-pH) procedures to measure the number of reflux events following ingestion of a refluxogenic meal (sausage and egg breakfast sandwich 8 oz. cup of coffee). The supervised dose was the only dose administered on this initial day of testing (Day 1). Subjects continued dosing for a further five day, then had one day with no study medication before returning for the next visit on Day 8. The same procedure was performed on Day 8 of the treatment period (i.e., the first day of the second dosing period).

A summary of the above described procedures to assess pharmacodynamic endpoints on Days 1 and 8 is set forth below:
Fasting 8 hours
Study medication administration;
Manometry: standard pull through and location of lower esophageal sphincter (45 minutes after study medication administration);
Measurement of resting LESP for 15 minutes (from 45 minutes to 60 minutes after study medication administration);
Removal of manometry catheter and insertion of MII-pH probe (one hour after study medication administration);
Refluxogenic meal;
MII/pH measurements for 2 hours (starting 5 minutes after meal completion).

In addition to assessment of the LESP and number of reflux events, subjects were also assessed on Days 1 and 8 to determine the frequency of the following symptoms: heartburn; regurgitation; and acid taste associated with the refluxogenic meal.

On Days 2-6 of the first dosing period and Days 8-13 of the second dosing period, subjects self-administered three doses of study medication at 0.5 to one hour prior to breakfast, lunch and dinner. On Day 7 (the day before the second assessment) no medication was taken to washout the week one treatment.

### Study Objectives:

Characterize the effect of DDP733 on esophageal-related pharmacodynamic measurements, including changes in reflux episodes, lower esophageal sphincter pressure and specific symptoms (heartburn, regurgitation, acid taste) in volunteers following ingestion of a refluxogenic meal.

Characterize the safety and tolerability of DDP733 in volunteers.

### Clinical Endpoint Measurements:

Primary: Reduction in esophageal reflux during a provocative procedure (following a refluxogenic meal) as measured by MII-pH;
Secondary: (a) Change in reflux related symptoms (heartburn, regurgitation, acid taste) associated with ingestion of a refluxogenic meal; and (b) Change in lower esophageal sphincter pressure.

### Results:

The FIGURE shows that a dose of 0.5 mg of DDP733 achieved statistical significance (p=0.0313) on the primary clinical endpoint of reduction in the number of reflux events versus placebo. Notably, this reduction in reflux events of about 40% is clinically meaningful. The doses of 0.8 mg and 1.4 mg did not show a statistically significant reduction in the number of reflux events versus placebo. Statistical analysis was performed using the Wilcoxon signed-rank test for paired data.

For the secondary endpoints of LESP and reflux related symptoms there were no statistically significant differences in treatment groups receiving DDP733 (0.5 mg, 0.8 mg and 1.4 mg) versus placebo.

### Safety Measurements:

Safety measurements included monitoring of vital signs and adverse events, clinical laboratory testing and performance of electrocardiograms (ECGs). DDP733 was safe and well tolerated at all doses. There were no significant adverse events (SAEs) reports and no adverse events (AEs) leading to discontinuation.

Drug related adverse events (nausea, vomiting, pruritus, rash, rash macular, flush/hot flush and abdominal pain/discomfort) were all mild/moderate, resolved within 1-5 days and did not require medication. No liver or cardiac abnormalities were observed.

While this invention has been particularly shown and described with references to example embodiments thereof, it will be understood by those skilled in the art that various changes in form and details may be made therein without departing from the scope of the invention encompassed by the appended claims.

1. A method of treating GERD in a human subject in need thereof comprising orally administering to said subject an effective amount of a compound represented by the following structure: 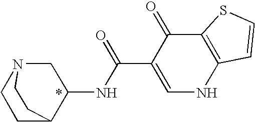
or a pharmaceutically acceptable salt thereof, wherein the effective amount is from about one to about three daily doses of the compound and the dose is from about 0.2 mg to about 0.5 mg.

2. The method of claim 1, wherein the subject suffers from n-GERD.

3. The method of claim 1, wherein the asterisked carbon atom is in the (R) configuration.

4. The method of claim 3, wherein the compound is in the form of the monohydrochloride salt.

5. The method of claim 1, wherein the compound is administered in a single daily dose.

6. The method of claim 5, wherein the dose is about 0.5 mg.

7. The method of claim 6, wherein the single dose is administered coincident with the subject's bedtime.

8. The method of claim 6, further comprising co-administering a proton pump inhibitor.

9. The method of claim 8, wherein the proton pump inhibitor is selected from the group consisting of: esomeprazole, omeprazole, lansoprazole, rabeprazole and pantoprazole.

10. The method of claim 1, wherein the compound in administered twice a day.

11. The method of claim 10, wherein the dose is about 0.5 mg.

12. The method of claim 11, further comprising co-administering a proton pump inhibitor.

13. The method of claim 12, wherein the proton pump inhibitor is selected from the group consisting of: esomeprazole, omeprazole, lansoprazole, rabeprazole and pantoprazole.

14. The method of claim 1, wherein the compound is administered three times a day.

15. The method of claim 14, wherein the dose is about 0.5 mg.

16. The method of claim 15, wherein the compound is administered coincident with the morning meal, midday meal and bedtime of the subject.

17. The method of claim 15, further comprising co-administering a proton pump inhibitor.

18. The method of claim 17, wherein the proton pump inhibitor is selected from the group consisting of: esomeprazole, omeprazole, lansoprazole, rabeprazole and pantoprazole.

19-32. (canceled)

33. A method of treating n-GERD in a human subject in need thereof comprising orally administering to the subject an effect amount of DDP-733 and a proton pump inhibitor, wherein 0.5 mg of the DDP-733 and the proton pump inhibitor are present in a single composition.

34. The method of claim 33, wherein the single composition comprises two distinct portions, wherein the first portion contains about 0.5 mg DDP733 and the second portion contains the proton pump inhibitor.

35-36. (canceled)
